SEQUENCE LISTING (5 sequences)

SEQ ID NO: 1 - DNA, 25 bp, Artificial
  synthetic primer
gtagtgaggaggcttttttggaggc

SEQ ID NO: 2 - DNA, 22 bp, Artificial
  synthetic primer
ccttcccgcttcagtgacaacg

SEQ ID NO: 3 - DNA, 29 bp, Artificial
  synthetic primer
aagcttgattgaacaagatggattgcacg

SEQ ID NO: 4 - DNA, 21 bp, Artificial
  synthetic primer
cggccgaaccccagagtcccg

SEQ ID NO: 5 - DNA, 821 bp, Artificial
  Amplified gene product
aagcttgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggccg

(19) United States
(12) Patent Application Publication
(10) Pub. No.: US 2014/0065669 A1
(43) Pub. Date: Mar. 6, 2014

(54) NEUROBLASTOMA HOST CELL LINE FOR STABLY EXPRESSING HUMAN EXOGENOUS PROTEIN AND METHOD

(71) Applicant: NATIONAL TAIWAN NORMAL UNIVERSITY, Taipei City (TW)

(72) Inventors: Guey-Jen LEE-CHEN, Taipei City (TW); Li-Ching LEE, Taipei City (TW); Chih-Hsin LIN, Yunlin County (TW)

(73) Assignee: National Taiwan Normal University, Taipei City (TW)

(21) Appl. No.: 13/858,272

(22) Filed: Apr. 8, 2013

(30) Foreign Application Priority Data
Sep. 6, 2012 (TW) 101132489

(51) Int. Cl. C07K 14/47; C12N 5/079

(52) U.S. Cl. CPC C07K 14/47; C12N 5/0618
USPC 435/69.1; 435/368

(57) ABSTRACT

A neuroblastoma host cell line for stably expressing human exogenous protein is provided. The cell line is generated by independently integrating control plasmid and switch plasmid into genome of human neuroblastoma SH-SY5Y cells. Upon inserting diseased gene into the above cell line, the cell line thus stably expresses the interested protein by induction, which can be used to study human neurological diseases related to the gene mutation.

### CROSS REFERENCE TO RELATED APPLICATION

This application claims the benefits of the Taiwan Patent Application Serial Number 101132489, filed on Sep. 6, 2012, the subject matter of which is incorporated herein by reference.

### BACKGROUND OF THE INVENTION

1. Field of the Invention

The present invention relates to a neuroblastoma host cell line for stably expressing human exogenous protein and, more particularly, to a neuroblastoma host cell line containing a Flp recombination target (FRT) site and a neomycin-resistance gene.

2. Description of Related Art

In vitro cell culture methods provide convenient and simple platforms to investigate functions or mechanisms of components derived from organisms. Recently, many biological experiments for in vitro cell culture methods are developed and improved, so the applications thereof can further be expanded. For example, the developed gene transfer techniques can be applied to biomedical studies.

The development of the cell cultures, and especially the human cell cultures can obtain experimental results fast, since the culture time thereof is shorter than that of animal experiments. In addition, compared to gene transfer techniques in microorganisms, desired proteins can be expressed in conditions similar to human physiological systems by using human cell culture methods. Hence, the human cell culture methods are applied widely to drug discoveries or researches on human diseases.

Generally, exogenous genes are inserted into chromosomes to continuously express exogenous proteins. However, proteins related to human diseases are cytotoxic, and the continuous expressions of these proteins may cause cell death. In addition, the different insertion sites and inserted gene numbers may cause the expressions of the exogenous proteins changed. Hence, cell lines without the aforementioned disadvantages have to be developed to establish systems which can be used to stably express specific human genes.

On the other hand, brain and spinal cord are composed of different neural cells (neurons), and synapses in different parts have different functions such as motor control, information processing, perception, learning and memories. Neurodegenerative diseases are symptoms that the functions of neuron in the brain and the spinal cord are lost. There are many neurodegenerative diseases found in clinic practice. For instance, dementia is one example of the neurodegenerative diseases which shows a problem of memory; and ataxia is another example thereof which shows a problem of lack of voluntary coordination of muscle movements.

Many ataxias fall under the category of poly-glutamine diseases (polyQ diseases) caused by CAG expansion in the disease genes. The polyQ diseases are autosomal dominant inheritance, and the onset ages and the symptoms of filial generations are earlier and more serious than those of parental generations. Recent studies on the polyQ diseases revealed that the abnormal expansion of glutamine is highly related to nine types of neurodegenerative diseases including Huntington's chorea and several types of spinocerebellar ataxias. Studies on the polyQ diseases found that the encoded polyQ proteins caused by CAG expansion cannot normally fold and has low solubility. In this case, the expanded polyQ proteins may accumulate in cells, and the accumulated proteins may affect the normal physiological functions of cells and cause neural cell death. Once the neural cells in the brain and the spinal cords are damaged or dead, the damaged or dead cells are difficult to regenerate. Hence, irreversibly permanent injuries may be formed after the brain or the spinal cords are damaged.

Since these kinds of neurodegenerative diseases are caused by expansion of the polyQ tract, researches are focused on discoveries of the mechanisms of polyQ formations and the treatments of these diseases. Many models are thus established, including cell models for stably encoding genes by using human cells. Nowadays, the well-established cell models for stably expressing human specific genes include human fetal kidney cell lines and human T-cell leukemia cell tines. The aforementioned cell models can be easily cultured and transfected, and therefore applied to various biological researches and biotechnology industries.

However, human fetal kidney cell lines and human T-cell leukemia cell lines are not neural cells, and these cell models still have their limitations in neurodegenerative disease studies. For example, the phenomenon similar to neural cells cannot be observed in the aforementioned cell models. Therefore, it is still desirable to provide a novel cell lines for stably expressing exogenous proteins, which can be applied to neurodegenerative disease studies.

### SUMMARY OF THE INVENTION

The present invention provides a neuroblastoma host cell line for expressing human exogenous proteins, which is human neuroblastoma cells transfected with a control plasmid containing Tet repressor gene and blasticidin-resistance gene, and a switch plasmid containing a Flp recombination target (FRT) site and a neomycin-resistance gene (no ATG). In the present invention, the control plasmid containing the Tet repressor gene and the blasticidin-resistance gene was transfected into human neuroblastoma cells such as SH-SY5Y cells, so 1-10 μg/mL blasticidin (preferably, 3-8 μg/mL) can be used to select the cells successfully transfected with the control plasmid. Furthermore, the switch plasmid used in the present invention is a plasmid containing the FRT site and the neomycin-resistance gene. The FRT site can provide a binding and cleavage site for the Flp recombinase. The neomycin-resistance gene is used to select the cells successfully transfected with the switch plasmid by using 100-1000 μg/mL G418 (preferably, 250-750 μg/mL). After transfecting the switch plasmid into human neuroblastoma cells, a PCR (polymerase chain reaction) process can be used to identify the FRT copy number of the switch plasmid. The example of the primer pair used for the PCR can be: 5′-GTAGTGAGGAGGCTTTTTTGGAGGC-3′ (SEQ ID NO:1) and 5′-CCTTCCCGCTTCAGTGACAACG-3′ (SEQ ID NO:2), but the present invention is not limited thereto. In addition, the neuroblastoma host cell line for expressing human exogenous proteins can be cultured in a medium containing 1-10 μg/mL blasticidin (preferably, 2-8 μg/mL) and 100-1000 μg/mL G418 (preferably, 250-750 μg/mL). The medium used in the present invention can be any medium generally used in the art as long as blasticidin and G418 contained therein, and depends upon the types or the species of the cells.

The human neuroblastoma SH-SY5Y host cell line of the present invention may be transfected with an expressing plasmid for expressing exogenous protein. Herein, the expressing plasmid preferably contains a human exogenous gene encoding human exogenous protein related to human neurological diseases. For example, the human exogenous gene is a gene related to human neurodegenerative diseases.

According to one aspect of the present invention, the neuroblastoma host cell line for expressing human exogenous proteins can be applied as follows. For example, an interest gene can be inserted into pcDNA5IFRT/TO containing a hygromycin-resistance gene (no ATG) to obtain a recombinant plasmid, and then the recombinant plasmid and a pOG44 are co-transfected into the aforementioned human neuroblastoma SH-SY5Y host cell line transfected with the control plasmid and the switch plasmid. After hygromycin selection, the cells successfully transfected with the recombinant plasmid can be obtained. Tetracycline or derivatives thereof can be used to induce the expression of the interest gene, and the concentration of tetracycline can be 1-10 μg/mL. In one embodiment of the present invention, the interest gene is TATA-box binding proteins (TBP) containing poly-glutamates. The abnormal expansion of the poly-glutamates may lead to spinocerebellar ataxia type 17 (SCA17) in human. Hence, the present invention provides an example to study SCA 17.

In addition, the neuroblastoma host cell line for expressing human exogenous proteins of the present invention can be used for not only studies of human diseases such as human neurodegenerative diseases, but also drug discoveries.

Other objects, advantages, and novel features of the invention will become more apparent from the following detailed description when taken in conjunction with the accompanying drawings.

### BRIEF DESCRIPTION OF THE DRAWINGS

FIG. 1A is a perspective view showing a construct of a control plasmid of the present invention;

FIG. 1B is a perspective view showing a construct of a switch plasmid of the present invention;

FIG. 2 is a perspective view showing a construct that an interest gene is inserted into pcDNA5/FRT/TO vector in one preferred embodiment of the present invention;

FIG. 3 is a perspective view showing an interest gene is induced with tetracycline; and

FIG. 4 is a result showing a TATA-box binding protein (TBP) with 36-79 glutamates expressed by the neuroblastoma host cell line for expressing human exogenous proteins according to one preferred embodiment of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Preparation of Neuroblastoma Host Cell Line for Stably Expressing Human Exogenous Proteins

[Transfection of Control Plasmid pcDNA6/TR]

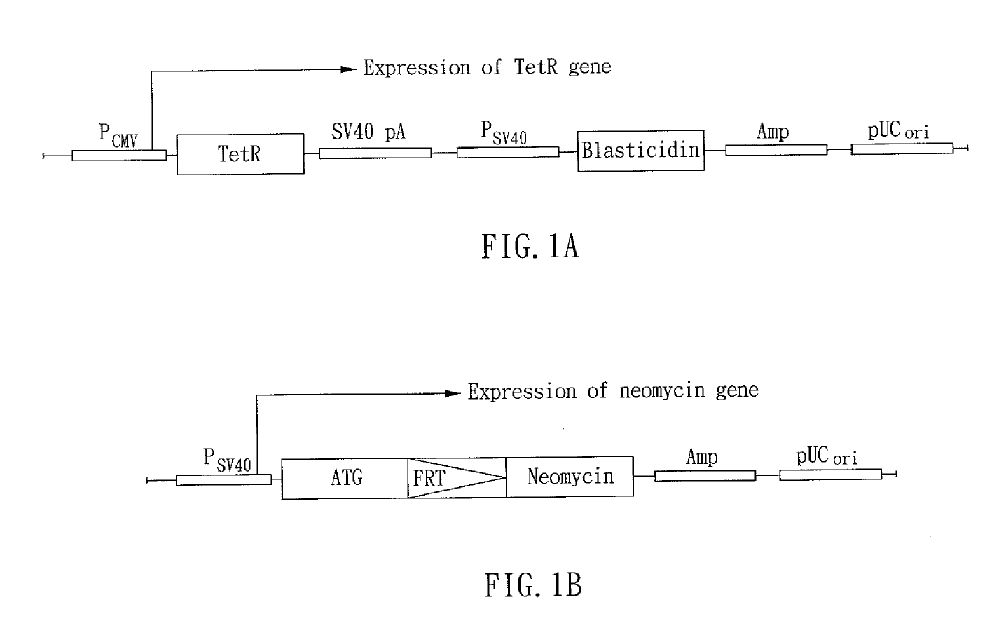

FIG. 1A is a perspective view showing a pcDNA6/TR (purchased from Invitrogen) used in the present embodiment, which is the control plasmid of the present invention. The pcDNA6/TR contains a TetR gene for expressing Tet repressor and a blasticidin-resistance gene. Herein, the pcDNA6/TR was transfected into human neuroblastoma SH-SY5Y cells by a liposome-mediated transfection method, and the derived cells were selected with 5 mg/mL of blasticidin.

### [Construction and Transfection of Switch Plasmid]

FIG. 1B is a perspective view showing a switch plasmid of the present embodiment. Neomycin open reading frame (ORF) without ATG was amplified with a PCR process. In the PCR process, pcDNA3 was used as a template, and the used primers were 5′-AAGCTTGATTGAACAAGATGGATTGCACG (SEQ ID NO: 3) and 5′-CGGCCGAACCCCAGAGTCCCG (SEQ ID NO: 4). After the PCR process, the amplified products shown in SEQ ID NO: 5 were cloned into pGEM-T Easy and verified by DNA sequencing. Next, 0.8 kb neomycin gene was removed with restriction enzymes HindIII and NotI therefrom, and used to replace IacZ-Zeocin gene in pFRT-lacZeo2 (Invitrogen). After ligation, a pFRT/neomycin plasmid containing a Flp recombination target (FRT) site was obtained, in which the FRT site was used as a binding and cleavage site for the Flp recombinase. In the present embodiment, the obtained pFRT/neomycin is the switch plasmid of the present invention.

SEQ ID NO: 5
AAGCTTGATTGAACAAGATGGATTGCACGCAGGTTCTCCGGCCGC
TTGGGTGGAGAGGCTATTCGGCTATGACTGGGCACAACAGACAATC
GGCTGCTCTGATGCCGCCGTGTTCCGGCTGTCAGCGCAGGGGCG
CCCGGTTCTTTTTGTCAAGACCGACCTGTCCGGTGCCCTGAATGAA
CTGCAGGACGAGGCAGCGCGGCTATCGTGGCTGGCCACGACGGG
CGTTCCTTGCGCAGCTGTGCTCGACGTTGTCACTGAAGCGGGAAG
GGACTGGCTGCTATTGGGCGAAGTGCCGGGGCAGGATCTCCTGTC
ATCTCACCTTGCTCCTGCCGAGAAAGTATCCATCATGGCTGATGCAA
TGCGGCGGCTGCATACGCTTGATCCGGCTACCTGCCCATTCGACCA
CCAAGCGAAACATCGCATCGAGCGAGCACGTACTCGGATGGAAGC
CGGTCTTGTCGATCAGGATGATCTGGACGAAGAGCATCAGGGGCT
CGCGCCAGCCGAACTGTTCGCCAGGCTCAAGGCGCGCATGCCCG
ACGGCGAGGATCTCGTCGTGACCCATGGCGATGCCTGCTTGCCGA
ATATCATGGTGGAAAATGGCCGCTTTTCTGGATTCATCGACTGTGGC
CGGCTGGGTGTGGCGGACCGCTATCAGGACATAGCGTTGGCTACC
CGTGATATTGCTGAAGAGCTTGGCGGCGAATGGGCTGACCGCTTC
CTCGTGCTTTACGGTATCGCCGCTCCCGATTCGCAGCGCATCGCCT
TCTATCGCCTTCTTGACGAGTTCTTCTGAGCGGGACTCTGGGGTT
CGGCCG

Underline: Primers as shown in SEQ ID NOs: 3 and 4
Italic: Neomycin open reading frame
Bold: Stop codon

After transfection with the pFRT/neomycin into the aforementioned human neuroblastoma SH-SY5Y cells containing the pcDNA6/TR control plasmid, cells from a single colony were selected with 500 μg/mL G418 antibiotics. The collected cells were continuously cultured in a medium containing 5 μg/mL blasticidin and 500 μg/mL G418.

The FRT copy numbers were determined by a PCR process using primers flanking FRT site: 5′-GTAGTGAGGAGGCTTTTTTGGAGGC-3′ (SEQ ID NO: 1) and 5′-CCTTCCCGCTTCAGTGACAACG-3′ (SEQ ID NO: 2). Cells with one copy FRT site was selected and used as a neuroblastoma host cell line for expressing human exogenous proteins of the present invention.

### Application of Neuroblastoma Host Cell Line for Expressing Human Exogenous Proteins

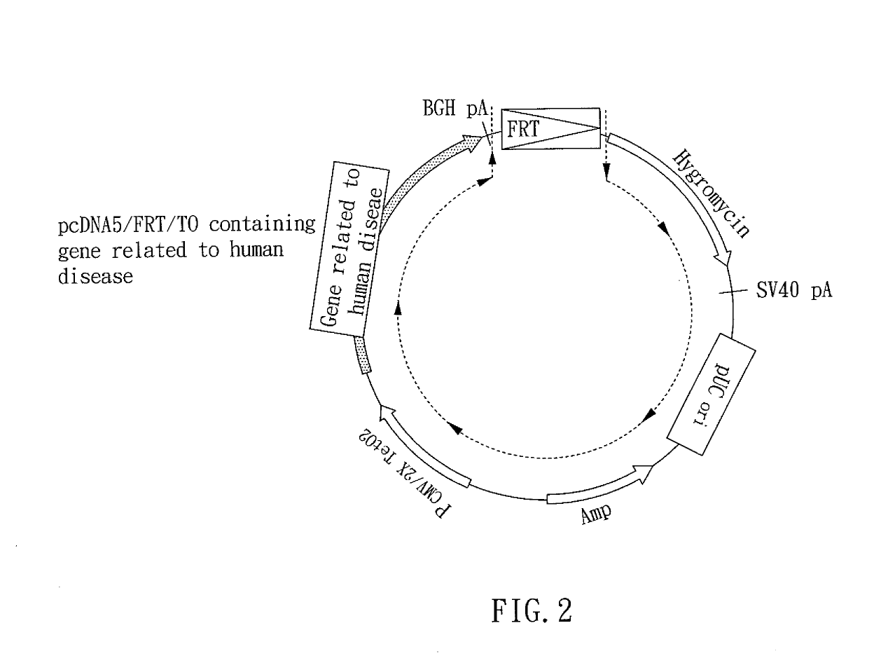

As shown in FIG. 2, an interest gene related to human disease was inserted into pcDNA5/FRT/TO (Invitrogen) to construct a recombinant plasmid. In the present embodiment, the interest gene is a DNA sequence which can encode 36-79 glutamates. The Q₃₆, Q₆₁ and Q₇₉cDNA in pGEM-T Easy vector (promega) was constructed as described by Lee L C et al. (Clin Chim Acta 400: 56-62, 2009; PLoS One 7: e35302, 2012), and cDNA of the interest gene were made by ligation Fnu4H1 partially digested fragments and the repeat number was verified by DNA sequencing. The recombinant plasmid and a pOG44 plasmid (Invitrogen) expressing the Flp recombinase were co-transfected into the above obtained SH-SY5Y neuroblastoma host cell line to construct isogenic TBP/Q_{36˜79} Cell lines.

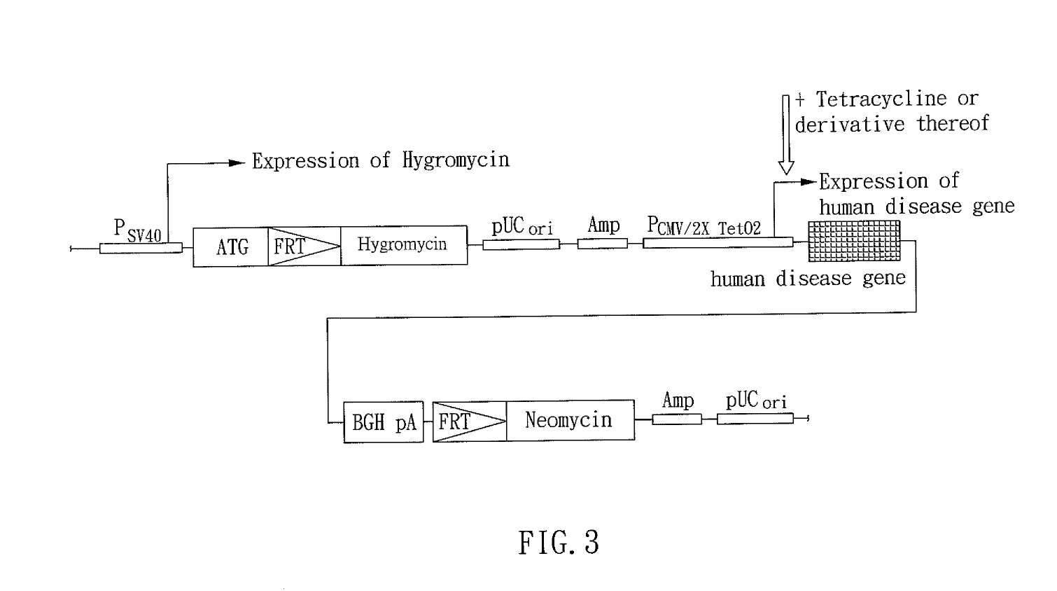

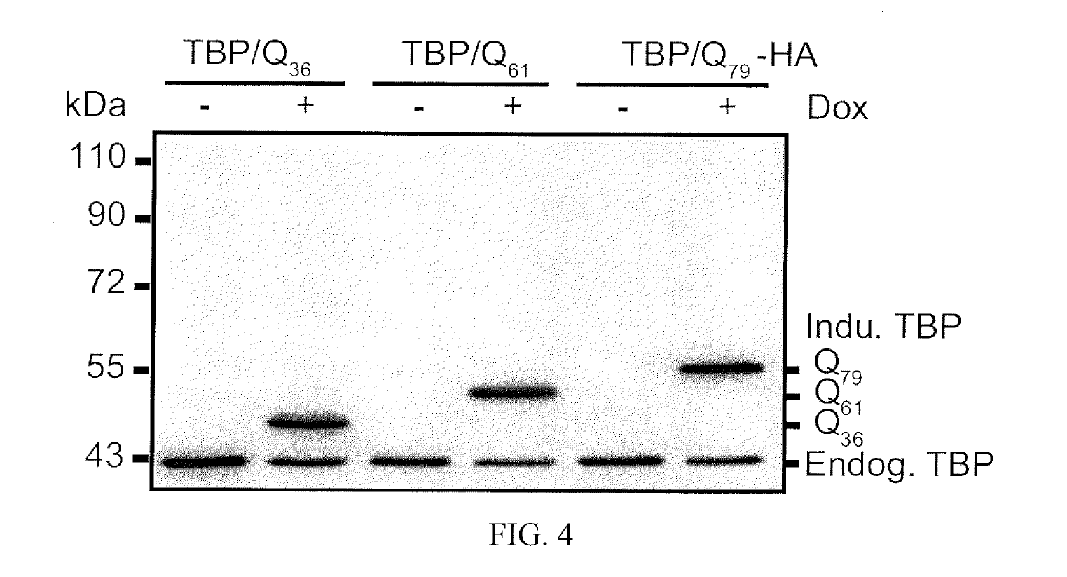

In the present embodiment, as shown in FIG. 3, 5 μg/mL tetracycline was added into the medium cultured with the co-transfected cells to induce the expression of TATA-box binding proteins (TBP) with 36˜79 glutamates. In addition, the expression of TBP can also be stopped by removing or without adding tetracycline, as shown in FIG. 4, in which the term “Dox” in this figure indicates tetracycline was added (+) or not added (−) to the medium for 6 days, the term “Indu. TBP” therein indicates the induced TBP protein, and the term “Endog. TBP” therein indicated the endogeneous TBP protein.

The above embodiments have been provided mainly for an illustrative purpose, they are not intended to be restricted by the above disclosure relating to protein expression, but can be applied in other proteins. Although the present invention has been explained in relation to its preferred embodiment, it is to be understood that many other possible modifications and variations can be made without departing from the spirit and scope of the invention as hereinafter claimed.

What is claimed is:

1. A neuroblastoma host cell line for expressing human exogenous protein, comprising:
human neuroblastoma cells;
a control plasmid, which is introduced into the human neuroblastoma cells by a transfection process and used to control an expression of human exogenous protein; and
a switch plasmid containing a Flp recombination target (FRT) site and a neomycin-resistance gene, wherein the switch plasmid is introduced into the human neuroblastoma cells by a transfection process.

2. The neuroblastoma host cell line as claimed in claim 1, wherein the human neuroblastoma cells are derived from human neuroblastoma SH-SY5Y cells.

3. The neuroblastoma host cell line as claimed in claim 1, wherein the control plasmid contains a blasticidin-resistance gene.

4. The neuroblastoma host cell line as claimed in claim 3, characterized in that the neuroblastoma host cell line is cultured in a medium containing blasticidin and G418.

5. A method for expressing human exogenous proteins, comprising:
(a) providing a neuroblastoma host cell line, which comprises:
human neuroblastoma cells; a control plasmid, which is introduced into the human neuroblastoma cells by a transfection process and used to control an expression of human exogenous protein; and a switch plasmid containing a Flp recombination target (FRT) site and a neomycin-resistance gene, wherein the switch plasmid is introduced into the human neuroblastoma cells by a transfection process;
(b) transfecting an expressing plasmid containing human exogenous gene into the human neuroblastoma cells.

6. The method as claimed in claim 5, wherein the human neuroblastoma cells are derived from human neuroblastoma SH-SY5Y cells.

7. The method as claimed in claim 5, wherein the control plasmid contains a blasticidin-resistance gene.

8. The method as claimed in claim 5, further comprising a step (c) after the step (b): culturing the human neuroblastoma cells transfected with the expressing plasmid.

9. The method as claimed in claim 8, wherein the human neuroblastoma cells transfected with the expressing plasmid is cultured in a medium containing blasticidin and G418.

10. The method as claimed in claim 5, further comprising a step (d) after the step (c): inducing the human exogenous gene in the expressing plasmid to express human exogenous proteins.

11. The method as claimed in claim 5, wherein the human exogenous gene is a gene related to human neurological diseases.

12. The method as claimed in claim 10, wherein the human exogenous gene is induced to express human exogenous proteins related to human neurological diseases.
